(19) United States
(12) Patent Application Publication
(10) Pub. No.: US 2019/0275188 A1
(43) Pub. Date: Sep. 12, 2019

(54) ELECTRICAL PLUG-INCORPORATED AEROSOL PERFUME DISPENSER

(71) Applicant: DONGGUAN YIH TEH ELECTRIC PRODUCTS CO., LTD., Dongguan City (CN)

(72) Inventor: Ming Jen Hsiao, Miaoli County (TW)

(21) Appl. No.: 16/162,374

(22) Filed: Oct. 16, 2018

Related U.S. Application Data
(63) Continuation-in-part of application No. 15/914,963, filed on Mar. 7, 2018.

(51) Int. Cl. A61L 9/14; A61L 9/12; B65D 83/14

(52) U.S. Cl. CPC A61L 9/14; A61L 2209/133; B65D 83/753; A61L 9/12

(57) ABSTRACT

An electrical plug-incorporated aerosol perfume dispenser that uses a negative pressure pipette in communication with a fragrance bottle and a gas delivery device to continuously deliver gas over the top side of the negative pressure pipette for causing a venturi effect to generate negative pressure adsorption in the negative pressure adsorption hole of the negative pressure pipette. Subject to this negative pressure adsorption effect, the molecules of the fluid fragrance are carried through the opposite drainage hole of the negative pressure pipette into the inside of an air duct around the negative pressure adsorption hole and delivered by the gas from the delivery device through an air outlet toward the outside open air. Based on the negative pressure adsorption structure, the electrical plug-incorporated aerosol perfume dispenser is safe in use.

This application is a Continuation-in-Part of application Ser. No. 15/914,963, filed Mar. 7, 2018.

### BACKGROUND OF THE INVENTION

### 1. Field of the Invention

The present invention relates to scent releasing devices and more specifically, to an electrical plug-incorporated aerosol perfume dispenser, which is based on a negative pressure structure.

### 2. Description of the Related Art

Perfume dispensers or aroma diffusers that are simple, safe and easy to use are products that are required by the market. Conventional perfume dispensers or aroma diffusers commonly use an oscillator to oscillate an aromatic fluid into fine droplets that are then delivered into the outside air by a fan. A similar design is seen in U.S. Pat. No. 7,963,460, entitled “Detachable aromatic nebulizing diffuser” that needs to use at least two electrical devices. Further,

Further, there are perfume dispensers or aroma diffusers that use a heating element such as an electric resistance heating element to heat an aromatic substance for producing a fragrance. For example, U.S. Pat. No. 8,201,957, entitled “Lamp-based scent releasing system” teaches the use of a lamp bulb for heating an aromatic substance to release a scent. However, electric resistance heating to heat an essential oil or a fragrance wax must consider the problem of electric safety. Furthermore, the sockets on the wall or in various places have different angle orientation problems. These angle orientation problems must be overcome to avoid fluid fragrance from pouring out.

### SUMMARY OF THE INVENTION

The present invention has been accomplished under the circumstances in view. It is therefore an object of the present invention to provide an electrical plug-incorporated aerosol perfume dispenser, which facilitates application in a safe manner.

To achieve this and other objects of the present invention, an electrical plug-incorporated aerosol perfume dispenser comprises a housing, an electrical plug assembly, a gas delivery device, an intermediate connection block, air duct and a negative pressure pipette. The housing comprises an accommodation chamber and an extension portion. The accommodation chamber is defined inside the housing. The extension portion extends horizontally from the top side of the housing, defining a plug hole in one side thereof. The electrical plug assembly is mounted in the accommodation chamber of the housing at one side. The gas delivery device is electrically connected to the electrical plug assembly. The intermediate connection block is mounted on the top side of the housing, comprising a first chamber and a second chamber. The first chamber has one side thereof provided with a first communication hole, and an opposite side thereof disposed in communication with the gas delivery device. The second chamber has one side thereof provided with a second communication hole, and an opposite side thereof disposed in communication with the plug hole. The air duct has one side thereof disposed in communication with the first communication hole, and a free opposite side thereof provided with an air outlet. The negative pressure pipette has a negative pressure adsorption hole located on one side thereof and disposed in communication with the air duct, and an opposite side thereof terminating in a drainage hole and disposed in communication with the second communication hole.

Further, the electrical plug-incorporated aerosol perfume dispenser is used with a fragrance bottle. The fragrance bottle comprises a bottle mouth detachably inserted into the plug hole, and a fluid fragrance contained therein and disposed in communication with the second chamber via the bottle mouth. The fluid fragrance contains general or volatile aromatic liquids. The electrical plug assembly provides electricity to the gas delivery device. The gas delivery device continuously delivers gas through the first chamber, the first communication hole to the air duct toward the air outlet. During the transportation of the gas by the gas delivery device, the gas continuously flows at a high speed above the negative pressure adsorption hole of the negative pressure pipette, causing a reduction in pressure around the negative pressure adsorption hole. This venturi effect generates negative pressure adsorption in the negative pressure adsorption hole. Subject to this negative pressure adsorption effect, the molecules of the fluid fragrance are carried through the drainage hole of the negative pressure pipette into the inside of the air duct around the negative pressure adsorption hole and delivered by the gas from the delivery device through the air outlet toward the outside open air.

The application of the electrical plug-incorporated aerosol perfume dispenser of the present invention does not need to use an electrical heater to heat the fluid fragrance or an oscillator to oscillate the fluid fragrance into a mist of fluid droplets. The electrical plug-incorporated aerosol perfume dispenser uses the venturi effect of the structure, that is, the sustainable output produces an aromatic odor, which is safe to use.

The user can easily pull out the exhausted fragrance bottle out of the plug hole, and then use a new or different flavor of fragrance bottle, and then open the bottle cap from the bottle mouth and insert the bottle mouth into the plug hole of the extension portion of the housing. This design facilitates quick displacement of the fragrance bottle to enjoy the atmosphere of the aromatic gas or to alleviate the odor in the open space.

It is another object of the present invention to provide an electrical plug-incorporated aerosol perfume dispenser, which can be installed in an electrical socket on the wall and rotated relative to the electrical socket to one of a series of angular positions within a predetermined range of rotation to prevent the fragrance from being turned upside down where the contained fluid fragrance can be poured out.

To achieve this and other objects of the present invention, the electrical plug assembly of the electrical plug-incorporated aerosol perfume dispenser is a rotary plug structure comprising a pressure ring fixedly mounted in the locating hole of the housing and a rotary plug rotatably mounted within the pressure ring and electrically connectable to an external electrical socket. The pressure ring comprises a plurality of engaging protrusions spaced around an inner perimeter thereof, and a plurality of buffer notches located on and spaced around the periphery thereof. The rotary plug comprises a plurality of retaining grooves spaced around the periphery thereof and selectively engaged with the engaging protrusions to secure the rotary plug to the pressure ring in one of a series of angular positions. In application, the rotary plug can be inserted into an electrical socket on a wall or at any place where electricity is available. At this time, the user can rotate the housing with the pressure ring relative to the rotary plug to the desired angular position. Further, the design of the buffer notches enhances the flexibility of the pressure ring, facilitating rotation and positioning of the rotary plug in the pressure ring.

### BRIEF DESCRIPTION OF THE DRAWINGS

FIG. 1 is an oblique top elevational view of an electrical plug-incorporated aerosol perfume dispenser in accordance with the present invention.

FIG. 2 is an exploded view of the electrical plug-incorporated aerosol perfume dispenser in accordance with the present invention.

FIG. 3 is a front view of the of an electrical plug-incorporated aerosol perfume dispenser in accordance with the present invention.

FIG. 4 is a sectional view taken in an enlarged scale along line A-A of FIG. 3.

### DETAILED DESCRIPTION OF THE PREFERRED EMBODIMENT

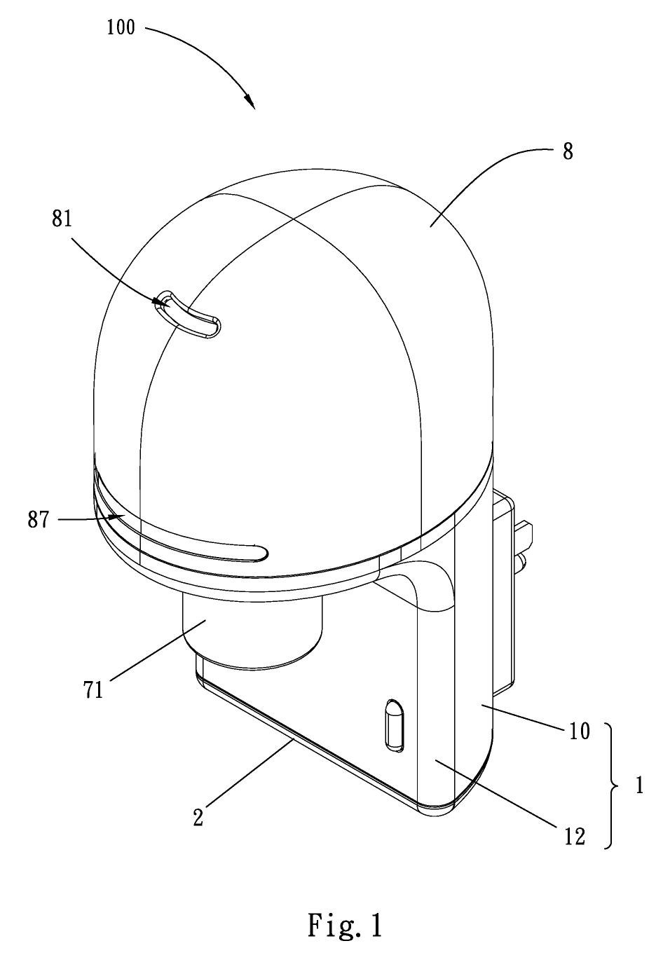

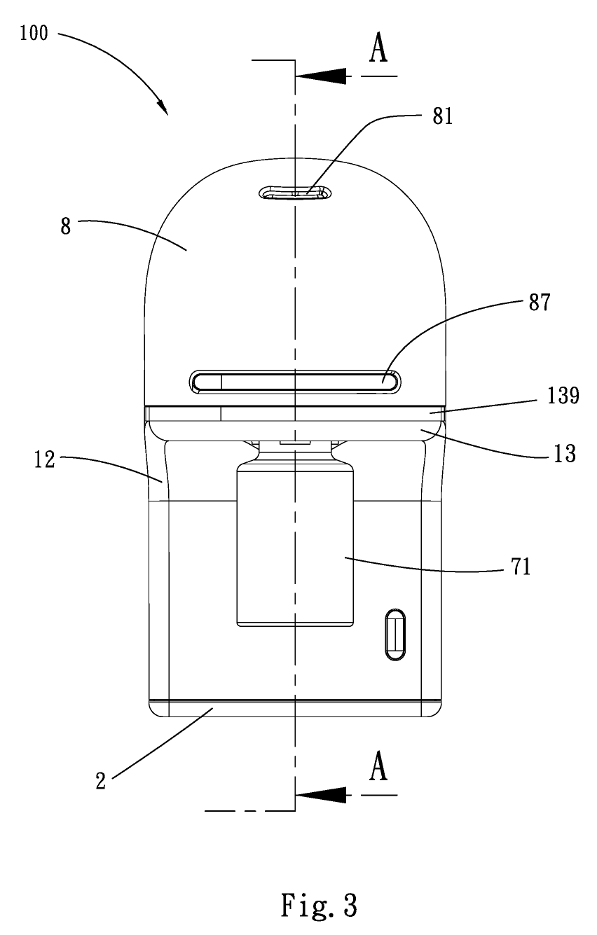

Referring to FIGS. 1-4, an electrical plug-incorporated aerosol perfume dispenser (100) in accordance with the present invention is shown. The aroma diffuser (100) comprises a housing (1), an electrical plug assembly (3), a gas delivery device (5), an intermediate connection block (7), an air duct (76) and a negative pressure pipette (78).

The housing (1) comprises an accommodation chamber (11) therein, an extension portion (13) horizontally extended from a top side thereof, a locating hole (15) located on the periphery, and a plug hole (131) located at the extension portion (13). The accommodation chamber (11) is not completely enclosed and allows the gas delivery device (5) to extract external air.

The electrical plug assembly (3) is mounted in the accommodation chamber (11) of the housing (1) at one side.

The gas delivery device (5) is electrically coupled with the electrical plug assembly (3) and used to provide continuous inhalation and delivery of gas.

The intermediate connection block (7) is mounted on the top side of the housing (1), comprising a first chamber (72) that accommodates the gas delivery device (5) and disposes in communication with the accommodation chamber (11), a second chamber (74) disposed in communication with the plug hole (131), a first communication hole (721) connected with the first chamber (72) and a second communication hole (741) connected with the second chamber (74).

The air duct (76) has one end thereof disposed in communication with the first communication hole (721), and an opposing free end thereof terminating in an air outlet (761). The negative pressure pipette (78) has a negative pressure adsorption hole (781) located at one end thereof and disposed in communication with the air duct (76), and a drainage hole (783) located at an opposite end thereof and disposed in communication with the second communication hole (741).

The electrical plug-incorporated aerosol perfume dispenser (100) is used with a fragrance bottle (71). The fragrance bottle (71) contains therein a fluid fragrance (713), comprising a bottle mouth (711) for plugging into the plug hole (131) to keep the contained fluid fragrance (713) in communication with the second chamber (74). The fluid fragrance (713) contains general or volatile aromatic liquids. The electrical plug assembly (3) provides electricity to the gas delivery device (5). The gas delivery device (5) continuously delivers gas through the first chamber (72), the first communication hole (721) to the air duct (76) toward the air outlet (761). During the transportation of the gas by the gas delivery device (5), the gas continuously flows at a high speed above the negative pressure adsorption hole (781) of the negative pressure pipette (78), causing a reduction in pressure around the negative pressure adsorption hole (781). This venturi effect generates negative pressure adsorption in the negative pressure adsorption hole (781). Subject to this negative pressure adsorption effect the molecules of the fluid fragrance (713) are carried through the drainage hole (783) of the negative pressure pipette (78) into the inside of the air duct (76) around the negative pressure adsorption hole (781) and delivered by the gas from the delivery device (5) through the air outlet (761) toward the outside open air.

The electrical plug-incorporated aerosol perfume dispenser (100) of the present invention does not need to use an electrical heater to heat the fluid fragrance or an oscillator to oscillate the fluid fragrance into a mist of fluid droplets. The electrical plug-incorporated aerosol perfume dispenser (100) uses the venturi effect of the structure, that is, the sustainable output produces an aromatic odor, which is safe to use.

The user can easily pull out the exhausted fragrance bottle (71) out of the plug hole (131), and then use a new or different flavor of fragrance bottle (71), and then open the bottle cap from the bottle mouth (711) and insert the bottle mouth (711) into the plug hole (131) of the extension portion (13) of the housing (1). This design facilitates quick displacement of the fragrance bottle (71) to enjoy the atmosphere of the aromatic gas or to alleviate the odor in the open space.

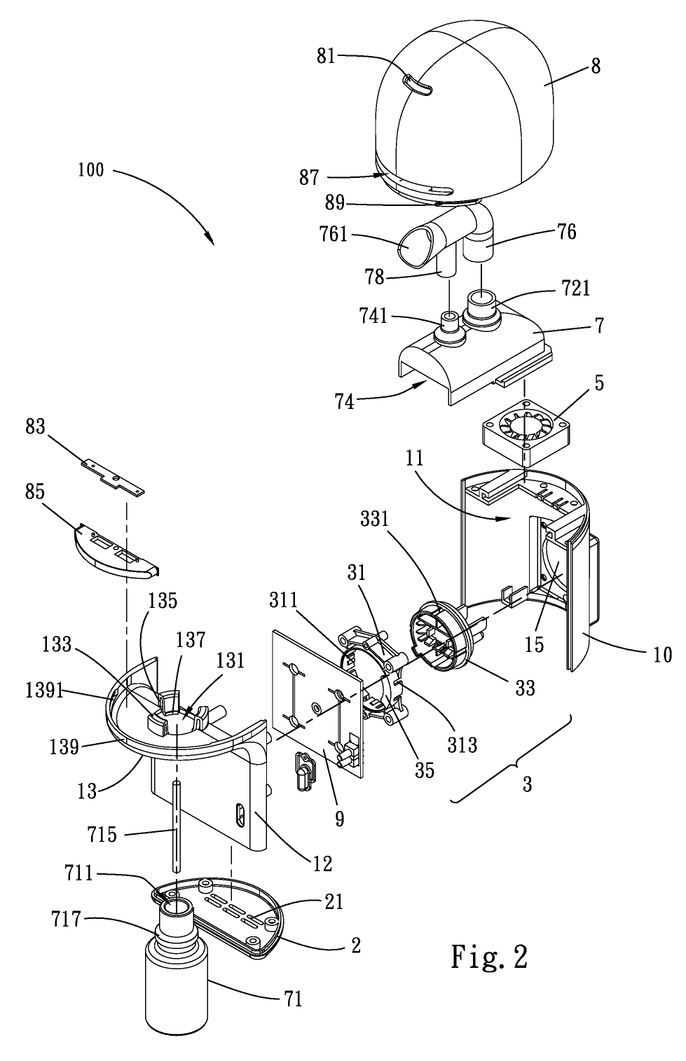

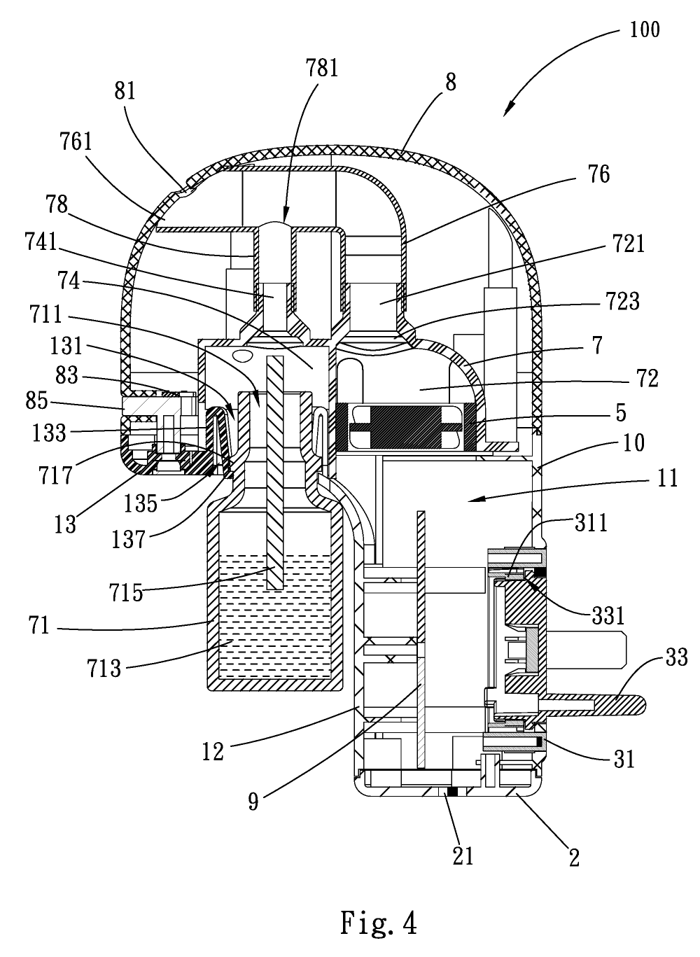

Referring to FIG. 2 and FIG. 4, in one embodiment of the present invention, the electrical plug assembly (3) is a rotary plug structure comprising a pressure ring (31) and a rotary plug (33). The pressure ring (31) is fixedly mounted in the locating hole (15), comprising a plurality of engaging protrusions (311) spaced around the inner perimeter thereof and respectively located at, for example, 90° angle, 180° angle and 270° angle, and a plurality of buffer notches (313) equiangularly spaced around the periphery. The rotary plug (33) comprises a plurality of retaining grooves (331) spaced around the periphery thereof for the engagement of the engaging protrusions (311). The rotary plug (33) is rotatably mounted within the pressure ring (31) such that the retaining grooves (331) can be shifted with the rotary plug (33) relative to the pressure ring (31) and engaged with the engaging protrusions (311) to secure the rotary plug (33) to the pressure ring (31) in one of a series of angular positions. The rotary plug (33) is electrically coupled with the gas delivery device (5) by, for example, an electrical line. In application, the rotary plug (33) can be inserted into an electrical socket on a wall or at any place where electricity is available. At this time, the user can rotate the housing (1) with the pressure ring (31) relative to the rotary plug (33) to the desired angular position, for example, 90°, 180° or 270° angular position. The design of the buffer notches (313) enhances the flexibility of the pressure ring (31), facilitating rotation and positioning of the rotary plug (33) in the pressure ring (31).

Referring to FIG. 2 and FIG. 4, a locating block (35) is disposed within the pressure ring (31) at a location between 270° and 360° angle. Subject to the arrangement of the locating block (35), the engaging protrusions (311) can simply be rotated with the pressure ring (31) relative to the rotary plug (33) within a predetermined range of rotation, for example, from 0° angle to 270° angle, preventing the fragrance bottle (71) from being turned upside down where the bottle mouth (711) faces down and the contained fluid fragrance (713) can be poured out of the fragrance bottle (71).

The fluid fragrance (713) can be essential oil, essence, flower essence, perfume or fragrance. The fluid fragrance is preferable a volatile liquid, or a fluid fragrance added with a volatile agent.

Referring to FIGS. 2 and 4 again, in the electrical plug-incorporated aerosol perfume dispenser (100), the radius of the negative pressure pipette (78) and/or the negative pressure adsorption hole (781) is shorter than the radius of the air duct (76).

The air is transported through the larger diameter of the air duct (76) over the smaller diameter of the negative pressure pipette (78) to generate a vacuum adsorption effect.

In one embodiment of the present invention, the negative pressure pipette (78) of the electrical plug-incorporated aerosol perfume dispenser (100) can be designed as a shrink tube structure (not shown). The narrowest part of the shrink tube structure forms the negative pressure adsorption hole (781). The negative pressure adsorption hole (781) of the shrink tube structure accelerates the delivery of the fluid fragrance molecules of the fluid fragrance (713) from the negative pressure adsorption hole (781) toward the air outlet (761) of the air dust (76).

In one embodiment of the present invention, as shown in FIGS. 2 and 4, an elastic retainer (133) is disposed around the plug hole (131) to secure the inserted bottle mouth (711) of the fragrance bottle (71). Thus, the bottle mouth (711) of the fragrance bottle (71) can be smoothly inserted into or removed from the plug hole (131).

In one embodiment of the present invention, as shown in FIGS. 2 and 4, the elastic retainer (133) comprises a U-shaped wall structure (135) and a protrusion (137). The U-shaped wall structure (135) is formed around the plug hole (131). The protrusion (137) protrudes from the free end of the U-shaped wall structure (135).

In the aforesaid embodiments, the elastic retainer (133) is selected from any of various elastic materials such as plastic materials, metal materials, metal sheets, fiber sheets or rubber. The elastic restoring energy of the elastic material of the elastic retainer (133) and its U-shaped wall structure (135) allows the aperture of the plug hole (131) to be elastically snapped on the bottle mouth (711) of the fragrance bottle (71).

In the aforesaid embodiments, in order to fit the cylindrical structure of the fragrance bottle (71), the plug hole (131) can be designed as a circular jack, and the U-shaped wall structure (135) and the protrusion (137) are circularly embodied.

In one embodiment of the present invention, as shown in FIGS. 2 and 4, the first chamber (72) and the first communication hole (721) form an inverted tapered structure (723), and the wide side of the inverted tapered structure (723) is connected to the first chamber (72), and the narrow side of the inverted tapered structure (723) is connected to the first communication hole (721). Thus, the gas continuously blown by the gas delivery device (5) is accumulated in the large space of first chamber (72) and then delivered by the inverted tapered structure (723) into the relatively smaller space of air duct (76) to accelerate the fluid velocity output so that when the gas flows over the top side of the negative pressure adsorption hole (781), a better negative pressure adsorption effect is produced.

In one embodiment of the present invention, as shown in FIGS. 2 and 4, the air duct (76) is an L-shaped tube structure; the negative pressure adsorption hole (781) of the negative pressure pipette (78) is joined to the inner side of the L-shaped tube structure of the air duct (76). The L-shaped tube structure of the air duct (76) shortens the length of the air duct (76) for installation in the limited device space.

In one embodiment of the present invention, as shown in FIGS. 1-4, the electrical plug-incorporated aerosol perfume dispenser (100) further comprises a hollow top cover (8) used as a decoration and protection of internal device components of the electrical plug-incorporated aerosol perfume dispenser (100). The hollow top cover (8) defines a fragrance outlet (81). The hollow top cover (8) is covered on the top side of the housing (1) over the intermediate connection block (7), the air duct (76) and the negative pressure pipette (78) to keep the fragrance outlet (81) in communication with the air outlet (761).

In one embodiment of the present invention, as shown in FIGS. 2 and 4, the electrical plug-incorporated aerosol perfume dispenser (100) further comprises a light source (83) and a light guide panel (85). The hollow top cover (8) has a mounting seat (87) located at one side thereof. The light source (83) is mounted in an inner side of the mounting seat (87). The light guide panel (85) is mounted in the mounting seat (87) and abutted to an outer side of the light source (83) with a part thereof disposed outside the mounting seat (87) (the outer perimeter of the hollow top cover). The light source (83) is electrically connected to the electrical plug assembly (3). The electrical plug assembly (3) provides electrical power to the light source (83) for emitting light. The light guide panel (85) absorbs the emitted light of the light source (83) and guides the incident light toward the outside of the hollow top cover (8) to create a lighting effect that matches the aroma atmosphere.

In one embodiment of the present invention, as shown in FIGS. 1-4, the housing (1) has a fence (139) formed on the top side of the extension portion (13). The fence (139) defines therein a retaining hole (1391). The hollow top cover (8) provides a retaining block (89). By means of fastening the retaining block (89) to the retaining hole (1391), the hollow top cover (8) is secured to the fence (139) of the housing (1).

In one embodiment of the present invention, as shown in FIGS. 1-4, the housing (1) consists of a first shell member (10) and a second shell member (12). The extension portion (13) horizontally extends from a top side of the second shell member (12) of the housing (1). The first shell member (10) has a space therein to define the accommodation chamber (11). The locating hole (15) is formed in the second shell member (12). The second shell member (12) is fastened to the first shell member (10) to cover the accommodation chamber (11).

In one embodiment of the present invention, as shown in FIGS. 2 and 4, the electrical plug-incorporated aerosol perfume dispenser (100) further comprises a control unit (9). The control unit (9) is mounted in the accommodation chamber (11) and electrically coupled with the electrical plug assembly (3), the light source (83) and the gas delivery device (5). The control unit (9) is used to control the electrical plug assembly (3), to electrically control the gas delivery device (5) to operate or stop, or to control the light source (83) to be turned on or off.

In one embodiment of the present invention, as shown in FIGS. 1-4, the electrical plug-incorporated aerosol perfume dispenser (100) further comprises a base plate (2) fastened to an opposing bottom side of the housing (1). The base plate (2) defines therein an air hole (21) for enabling outside air to flow into the inside of the housing (1).

In one embodiment of the present invention, as shown in FIGS. 1-4, the electrical plug-incorporated aerosol perfume dispenser (100) is mounted with a fragrance bottle (71), The fragrance bottle (71) further comprises a flange (717) extending around the bottle mouth (711). When inserting the bottle mouth (711) of the fragrance bottle (71) into the plug hole (131), the double layer wall of the U-shaped wall structure (135) can be elastically contracted to temporarily expand the aperture of the plug hole (131) for the passing of the flange (717) smoothly through the protrusion (137). Thereafter, the elastic retainer (133) of the U-shaped wall structure (135) immediately returns to its former shape, at the same time, the protrusion (137) is elastically clamped at the bottleneck on the lower side of the flange (717), thereby firmly securing the fragrance bottle (71) in the plug hole (131). Once the fluid fragrance (713) is exhausted, the user can conveniently and quickly withdraw the fragrance bottle (71) from the plug hole (131). At this time, the flange (717) compresses the protrusion (137) and the U-shaped wall structure (135) contracts to expand the plug hole (131), and thus the flange bottle (717) of the fragrance bottle (71) is quickly separated from the plug hole (131), so that the user can quickly and easily insert a new or different flavor of fragrance bottle (71) into the plug hole (131) for replacement.

In one embodiment of the present invention, as shown in FIGS. 1-4, the fragrance bottle (71) further comprises a diffusion unit (715). The diffusion unit (715) has one side thereof inserted through the bottle mouth (711) into the fluid fragrance (713) in the fragrance bottle (71). The diffusion unit (715) is used to absorb the fluid fragrance (713) and to accelerate the diffusion of the aromatic liquid molecules. The diffusion unit (715) has an opposite side thereof connected to the second chamber (74) and/or the second communication hole (741). The fluid fragrance (713) absorbed by the diffusion unit (715) is accelerated to be emitted (or volatilized). The fluid fragrance (713) can be a volatile aromatic liquid or a general aromatic liquid. The diffusion unit (715) facilitates the acceleration of the emission of the of aromatic molecules.

The aroma gas (containing fine aromatic water molecules) of the fluid fragrance (713) adsorbed by the diffusion unit (715) is delivered to the air outlet (761) by the negative pressure adsorption effect around the negative pressure adsorption hole (781) of the electrical plug-incorporated aerosol perfume dispenser (100) and then dissipated into the space outside the electrical plug-incorporated aerosol perfume dispenser (100).

The diffusion unit (715), such as cotton core, cotton swab, absorbent cloth, fiber rod, foam material, etc., can absorb the fragrance fragrances (713) and can emit various liquid fragrance molecules. In some embodiments of the present invention, the diffusion unit (715) is rod-shaped or strip-shaped to facilitate insertion through the bottle mouth (711) into the fluid fragrance (713).

Although particular embodiments of the present invention have been described in detail for purposes of illustration, various modifications and enhancements may be made without departing from the spirit and scope of the invention. Accordingly, the invention is not to be limited except as by the appended claims.

What the invention claimed is:

1. An electrical plug-incorporated aerosol perfume dispenser (100), comprising:
a housing (1), said housing (1) comprising an accommodation chamber (11), an extension portion (13) and a locating hole (15), said accommodation chamber (11) being defined inside said housing (1), said extension portion (13) extending horizontally from a top side of said housing (1) and defining a plug hole (131) in one side thereof;
an electrical plug assembly (3) mounted in said accommodation chamber (11) of said housing (1) at one side with the free end thereof extended out of said housing (1) through said locating hole (15);
a gas delivery device (5) electrically connected to said electrical plug assembly (3);
an intermediate connection block (7) mounted on the said top side of said housing (1), said intermediate connection block (7) comprising a first chamber (72) and a second chamber (74), said first chamber (72) having one side thereof provided with a first communication hole (721) and an opposite side thereof disposed in communication with said gas delivery device (5), said second chamber (74) having one side thereof provided with a second communication hole (741) and an opposite side thereof disposed in communication with said plug hole (131);
air duct (76) having one side thereof disposed in communication with said first communication hole (721) and a free opposite side thereof provided with an air outlet (761); and
a negative pressure pipette (78) having a negative pressure adsorption hole (781) located on one side thereof and disposed in communication with said air duct (76) and an opposite side thereof terminating in a drainage hole (783) and disposed in communication with said second communication hole (741).

2. The electrical plug-incorporated aerosol perfume dispenser (100) as claimed in claim 1, wherein said electrical plug assembly (3) is a rotary plug structure comprising a pressure ring (31) fixedly mounted in said locating hole (15) of said housing (1) and a rotary plug (33) rotatably mounted within said pressure ring (31) and electrically connectable to an external electrical socket, said pressure ring (31) comprising a plurality of engaging protrusions (311) spaced around an inner perimeter thereof and a plurality of buffer notches (313) located on and spaced around the periphery thereof, said rotary plug (33) comprising a plurality of retaining grooves (331) spaced around the periphery thereof and selectively engaged with said engaging protrusions (311) to secure said rotary plug (33) to said pressure ring (31) in one of a series of angular positions.

3. The electrical plug-incorporated aerosol perfume dispenser (100) as claimed in claim 2, wherein said electrical plug assembly (3) further comprises a locating block (35) disposed within said pressure ring (31) to limit the relative range of rotation between said pressure ring (31) and said rotary plug (33).

4. The electrical plug-incorporated aerosol perfume dispenser (100) as claimed in claim 1, wherein the radius of said negative pressure pipette (78) and/or said negative pressure adsorption hole (781) is shorter than the radius of said air duct (76).

5. The electrical plug-incorporated aerosol perfume dispenser (100) as claimed in claim 1, wherein one side of said negative pressure adsorption hole (781) is a shrink tube structure, and the narrowest part of said shrink tube structure forms said negative pressure adsorption hole (781).

6. The electrical plug-incorporated aerosol perfume dispenser (100) as claimed in claim 1, wherein said plug hole (131) is incorporated with an elastic retainer (133), said elastic retainer (133) being disposed around said plug hole (131).

7. The electrical plug-incorporated aerosol perfume dispenser (100) as claimed in claim 6, wherein said elastic retainer (133) comprises a U-shaped wall structure (135) surrounding said plug hole (131), and a protrusion (137) protruded from a free end of said U-shaped wall structure (135).

8. The electrical plug-incorporated aerosol perfume dispenser (100) as claimed in claim 1, further comprising a hollow top cover (8), said hollow top cover (8) comprising a fragrance outlet (81) disposed in communication with said air outlet (761) and a mounting seat (87) located at one lateral side thereof, said hollow top cover (8) being mounted on the said top side of said housing (1) to cover said intermediate connection block (7), said air duct (76) and said negative pressure pipette (78), wherein said electrical plug-incorporated aerosol perfume dispenser (100) further comprises a light source (83) and a light guide panel (85), said light source (83) being mounted in an inner side of said mounting seat (87) and electrically connected to said electrical plug assembly (3), said light guide panel (85) being mounted in said mounting seat (87) and abutted to an outer side of said light source (83).

9. The electrical plug-incorporated aerosol perfume dispenser (100) as claimed in claim 1, wherein said housing (1) consists a first shell member (10) and a second shell member (12), said extension portion (13) being horizontally extended from a top side of said second shell member (12), said first shell member (10) defining said accommodation chamber (11) therein, said second shell member (12) being fastened to said first shell member (10) to cover said accommodation chamber (11).

10. The electrical plug-incorporated aerosol perfume dispenser (100) as claimed in claim 1, wherein said air duct (76) is a L-shaped tube structure; said negative pressure adsorption hole (781) of said negative pressure pipette (78) is connected to an inner side of said L-shaped tube structure of said air duct (76).

11. The electrical plug-incorporated aerosol perfume dispenser (100) as claimed in claim 8, wherein said housing (1) further comprises a fence (139) located on a top side of said extension portion (13) and a retaining hole (1391) located in said fence (139); said hollow top cover (8) comprises a retaining block (89), which is fastened to said retaining hole (1391) to secure said hollow top cover (8) to said fence (139) of said housing (1).

12. The electrical plug-incorporated aerosol perfume dispenser (100) as claimed in claim 8, further comprising a control unit (9) mounted in said accommodation chamber (11) and electrically coupled with said electrical plug assembly (3), said light source (83) and said gas delivery device (5).

13. The electrical plug-incorporated aerosol perfume dispenser (100) as claimed in claim 1, further comprising a fragrance bottle (71), said fragrance bottle (71) comprising a bottle mouth (711) detachably inserted into said plug hole (131), a flange (717) extended around said bottle mouth (711) and engaged in said plug hole (131) and a fluid fragrance (713) contained therein and disposed in communication with said second chamber (74) via said bottle mouth (711).

14. The electrical plug-incorporated aerosol perfume dispenser (100) as claimed in claim 2, further comprising a fragrance bottle (71), said fragrance bottle (71) comprising a bottle mouth (711) detachably inserted into said plug hole (131), a flange (717) extended around said bottle mouth (711) and engaged in said plug hole (131) and a fluid fragrance (713) contained therein and disposed in communication with said second chamber (74) via said bottle mouth (711).

15. The electrical plug-incorporated aerosol perfume dispenser (100) as claimed in claim 4, further comprising a fragrance bottle (71), said fragrance bottle (71) comprising a bottle mouth (711) detachably inserted into said plug hole (131), a flange (717) extended around said bottle mouth (711) and engaged in said plug hole (131) and a fluid fragrance (713) contained therein and disposed in communication with said second chamber (74) via said bottle mouth (711).

16. The electrical plug-incorporated aerosol perfume dispenser (100) as claimed in claim 6, further comprising a fragrance bottle (71), said fragrance bottle (71) comprising a bottle mouth (711) detachably inserted into said plug hole (131), a flange (717) extended around said bottle mouth (711) and engaged in said plug hole (131) and a fluid fragrance (713) contained therein and disposed in communication with said second chamber (74) via said bottle mouth (711).

17. The electrical plug-incorporated aerosol perfume dispenser (100) as claimed in claim 13, wherein said fragrance bottle (71) further comprises a diffusion unit (715) inserted with one side thereof through said bottle mouth (711) into said fluid fragrance (713).

18. The electrical plug-incorporated aerosol perfume dispenser (100) as claimed in claim 14, wherein said fragrance bottle (71) further comprises a diffusion unit (715) inserted with one side thereof through said bottle mouth (711) into said fluid fragrance (713).

19. The electrical plug-incorporated aerosol perfume dispenser (100) as claimed in claim 15, wherein said fragrance bottle (71) further comprises a diffusion unit (715) inserted with one side thereof through said bottle mouth (711) into said fluid fragrance (713).

20. The electrical plug-incorporated aerosol perfume dispenser (100) as claimed in claim 16, wherein said fragrance bottle (71) further comprises a diffusion unit (715) inserted with one side thereof through said bottle mouth (711) into said fluid fragrance (713).
